(19) United States
(12) Patent Application Publication
(10) Pub. No.: US 2015/0265463 A1
(43) Pub. Date: Sep. 24, 2015

(54) OPHTHALMIC DEVICE FOR TREATING TISSUE IN THE ANTERIOR OF AN EYE

(71) Applicant: Carl Zeiss Meditec AG, Jena (DE)

(72) Inventors: Artur Hoegele, Oberkochen (DE); Christian Beder, Aalen (DE); Christoph Hauger, Aalen (DE); Joachim Steffen, Westhausen (DE); Michael Stefan Rill, Jena (DE)

(21) Appl. No.: 14/664,889

(22) Filed: Mar. 22, 2015

(30) Foreign Application Priority Data
Mar. 21, 2014 (DE) 10 2014 004 026.7

(51) Int. Cl. A61F 9/008

(52) U.S. Cl. CPC A61F 9/008

(57) ABSTRACT

An ophthalmic device for treating tissue in the anterior of an eye includes a laser for generating a light beam, an optical device for reshaping the light beam into a line focus, wherein a ratio of length to width of the line focus is at least 10, preferably 100, particularly preferably 1000, and an optical system for guiding the light beam to an object plane, in which the tissue to be treated is arrangeable, wherein the laser emits yellow light in a wavelength range between 525 nm and 675 nm, preferably between 550 nm and 610 nm, particularly preferably between 580 nm and 610 nm.

### CROSS REFERENCE TO RELATED APPLICATION

This application claims priority of German patent application no. 10 2014 004 026.7, filed Mar. 21, 2014, the entire content of which is incorporated herein by reference.

### FIELD OF THE INVENTION

The invention relates to an ophthalmic device for treating tissue in the anterior of an eye and comprises a laser for generating a light beam and an optical device for reshaping the light beam into a line focus. Here, a ratio of length to width of the line focus is at least 10, preferably 100, particularly preferably 1000. The ophthalmic device comprises an optical system for guiding the light beam to an object plane, in which the tissue to be treated is arrangeable.

### BACKGROUND OF THE INVENTION

Capsulorhexis is an example for an eye operation in the anterior of an eye. Here, a piece of the anterior capsular bag of an eye is scored in a circular region and opened, and the lens is removed through this hatch. The removed lens is replaced by an artificial lens or intraocular lens at the same position.

EP 0 467 775 B1 discloses a laser apparatus for cutting a lens capsule, having a device for generating a pulsed infrared laser beam and a device for projecting the laser beam onto a lens capsule in order to cut the latter. The projection device contains an optical focusing device for focusing the laser beam and an axicon lens device for projecting the focused beam in a ring-shaped form onto the lens capsule and for modifying the beam diameter. The focused laser beam on the aforementioned lens capsule has an energy density of no less than 10⁸ W/cm².

A disadvantage of the aforementioned laser apparatus is that the focused laser beam of the infrared laser has a very high energy density on the lens capsule and it is incident on the eye fundus at, or at least in the vicinity of, the macula. As a result, the region of the retina with the greatest density of visual cells is exposed to a particular risk. Even a short-term contact with the laser beam with this high energy density can lead to the destruction of tissue, even of the surrounding tissue region. This high energy density can also cause a critical situation for the operator. Particular safety measures are indispensable.

U.S. Pat. No. 8,562,596 B2 has described a laser-assisted method for complete or partial severing of tissue containing collagen. In one embodiment, the method relates to capsulorhexis, wherein the laser-assisted method is applied to the lens capsule. A light-absorption means is added to the tissue. A light ray with a wavelength suitable for being absorbed by the light-absorption means is directed onto the tissue in order to bring about a thermal effect.

A disadvantage of the aforementioned laser-assisted method is that a scanning system is required for the laser projection onto the location of the capsulorhexis. Such scanning systems are complex, voluminous and often not very economical. As a result of the comparatively long cutting duration, scanning systems are critical in the case of shaking or rolling of the eye during the cutting process. As a result of the comparatively long exposure time to the laser during the scanning cutting process, the tissue around the region to be cut is additionally heated by thermal dissipation.

### SUMMARY OF THE INVENTION

It is an object of the invention to provide a device for carrying out treatment of tissue in the anterior of an eye very quickly, with very high safety and with a protective treatment for the eye to be treated.

This object achieved by an ophthalmic apparatus for treating a tissue in the anterior of an eye. The ophthalmic apparatus includes: a laser configured to generate a light beam; an optical arrangement configured to reshape the light beam into a line focus having a length to width ratio of at least 10; an optical system configured to guide the light beam to an object plane configured to have the tissue to be treated arranged therein; and, the laser being configured to emit yellow light in a first wavelength range of 525 nm to 675 nm.

According to the invention, the object is achieved by virtue of the laser emitting yellow light in a first wavelength range between 525 nm and 675 nm, preferably between 550 nm and 610 nm, particularly preferably between 580 nm and 610 nm.

By reshaping a laser light beam into a light ray with a line-shaped cross section and the focusing into a line focus, it is possible to treat eye tissue in one work step (that is, without performing a scan) along the line determined by the line focus. In this way, it is possible to generate, for example, a tear or a cut in the tissue. Within the meaning of the present patent application, the term “line-shaped cross section” is understood to mean any line-shaped, straight or curved, closed or open, continuous or interrupted structures in general, the dimensions of which in the line direction are many times (for example, ten times, one hundred times or one thousand times) greater than across the line direction.

Focusing the light beam in the focal plane in which the tissue to be treated is also arranged brings about a high power density at the location of the line focus. If use is made of a laser with yellow light, thermal treatment of the tissue region can be achieved.

By providing the line focus and treating the eye tissue in one work step, a cut is carried out in a very short time, for example, less than one second, less than 500 ms or less than 250 ms. As a result of the very short exposure time of the eye to the laser, the risk of shaking is minimized. This results in a sharp cut contour. The very short exposure time of the laser is advantageous in that the heat dissipation to surrounding tissue is very low. This reduces the energy influx from the laser into the tissue of the eye. As a result, the cutting process can be carried out with the minimally required laser power. This increases the safety and brings about the sparing treatment of the eye.

In one embodiment of the invention, the numerical aperture and an image of the line focus in the object plane are matched to one another in such a way that the energy density in the object plane has a magnitude that is less than 10⁶ W/cm², preferably less than 10⁵ W/cm², particularly preferably less than 10⁴ W/cm².

A relatively low energy influx in relation to the area to be treated has greater safety for the eye and brings about the sparing treatment of the eye to be treated.

In one embodiment of the invention, the optical device for reshaping the light beam into a line focus includes a concave or convex axicon or a diffractive optical element or a reflection echelon grating or a micro-mirror array.

Concave or convex axicons, diffractive optical elements and reflection echelon gratings are members of a group of optical elements, via which light beams from lasers can easily be reshaped into light rays with round, oval or elliptical cross sections. Micro-mirror arrays, also known as “digital micro-mirror devices” (DMD), are constructed from many small switchable mirrors. With the aid thereof, it is possible to reshape light beams from lasers into light rays with virtually any cross section.

In one embodiment of the invention, the laser has a power maximum at a wavelength of 599 nm.

The power maximum of the yellow laser is advantageously matched to the absorption spectrum of the tissue to be treated. If the tissue to be treated has a maximum absorption behavior at 599 nm, a particularly efficient treatment of the tissue can be achieved if the power maximum of the laser is configured for this wavelength.

In one embodiment of the invention, the ophthalmic device includes an observation apparatus and a device for treating tissue in the anterior of an eye according to one of the preceding aspects.

Combining an observation apparatus with the ophthalmic device for treating tissue in the anterior of an eye enables the direct observation of the eye and the processes in the focal plane via the observation apparatus. The observation apparatus can be a surgical microscope. Advantageously, all functions of the surgical microscope, for example, a zoom function, a superposition of a target contour, surrounding or coaxial illumination, are additionally available to the user. A surgical microscope can provide an observation apparatus for a plurality of observers or surgeons. A surgical microscope can have a camera system with image processing. The latter can advantageously be used for monitoring the eye as a safety apparatus of the laser when treating the tissue of the eye.

In one embodiment of the invention, the observation apparatus has a main objective and a coupling element for coupling the light beam into the observation beam path.

When coupling the light beam of the laser or light beams of the line focus into the observation beam path, the optical axes of the imaging optical system for the light beam of the laser correspond to the optical axis of the observation beam path. This is advantageous in that the observation apparatus is easily focusable onto the focal plane of the laser apparatus such that processes in the focal plane can be immediately observed by the surgical microscope.

In one embodiment of the invention, the coupling element is embodied as a reflection bandpass filter, the bandpass transmission value of which is T>95% for a wavelength of between 599 nm+/−10 nm.

If the coupling element is embodied as a reflection bandpass filter, there is a high reflection of the laser light in the defined wavelength range, while the light from the other wavelength regions can pass the reflection bandpass filter almost without impediment. An advantage is that laser light reflected back from the eye is likewise deflected laterally by a bandpass transmission value of T>95%. As a result, the observer is reliably protected from back-reflected laser light.

In one embodiment of the invention, the coupling element is arranged above the main objective.

When arranging the coupling element above the main objective, the ophthalmic device for treating tissue can be integrated into the observation apparatus or into the surgical microscope. Components of the observation apparatus, for example, a control unit, can advantageously also be used for the ophthalmic device for treating tissue. The advantage is a more compact setup of the overall system. A further advantage is that the working distance between the main objective and the eye is maintained and so the whole working space below the main objective is available to the surgeon.

In one embodiment of the invention, the observation apparatus has ambient illumination or a coaxial illumination, from which fixation light is decoupled.

A fixation light is a light spot which the patient fixes on with his eye during an operation. As a result, the location of the patient eye is in a defined and stable position. The laser contour to be projected can easily be positioned on the eye. By decoupling the fixation light from the ambient illumination or the coaxial illumination of the observation apparatus, a very compact structure is possible since there is no need to provide a separate light source, including the actuation electronics required for this purpose. A fixation light can thus be integrated into the ophthalmic device in a very cost-effective and space-saving manner.

In one embodiment of the invention, fixation light is decoupled from the light beam of the laser.

A further option for integrating a fixation light into the ophthalmic device in a space-saving manner is to decouple a small portion of the laser light and using this as fixation light.

In one embodiment of the invention, a dye is introduced into the object plane of the tissue region by an ophthalmic device according to one of the aforementioned aspects in a method for thermal treatment of tissue in the anterior of an eye, which dye has a light-absorbing effect for the wavelength range of the laser.

This embodiment allows the tissue to be treated in a particularly sparing manner. To this end, the tissue to be treated is enriched with a dye bound to an extracellular matrix of the tissue or with a free dye, the absorption maximum of which dye lies in the emission spectrum of the laser. If the enriched tissue is irradiated by the laser beam, there is an excessive absorption in the tissue, via which the enriched, irradiated tissue is strongly heated locally without excessively damaging the adjacent tissue parts. As a result of the high absorptivity, thermal treatment of the tissue can thus already be achieved at a low laser power.

In one embodiment of the invention, the trypan blue dye is used, which has a maximum of the light-absorbing effect in the tissue region at a wavelength of 599 nm.

The laser is typically operated at an emission wavelength of 590 nm to 610 nm if trypan blue is used as a dye. If the power maximum of the laser is tuned to a wavelength of 599 nm, the dyed tissue can be treated thermally with a very high effectiveness with minimally required laser power. As a result, a very short treatment time can be achieved, which is connected to reduction in the risk of shaking when the eye rolls during the operation. This results in a very good cut result with little heating of the surrounding tissue. The operation can be performed in a particularly sparing manner for the eye as a result of the minimal energy influx into the eye.

### BRIEF DESCRIPTION OF THE DRAWINGS

The invention will now be described with reference to the drawings wherein:

FIG. 1 is a schematic of a first embodiment of an ophthalmic device with a laser apparatus according to the invention in an arrangement below a surgical microscope;

FIG. 2 is a schematic of a second embodiment of an ophthalmic device with a laser apparatus according to the invention in an arrangement in a surgical microscope;

FIG. 3 is a schematic of a third embodiment of an ophthalmic device with a laser apparatus according to the invention in an arrangement below a surgical microscope with a micro-mirror array;

FIG. 4 is a schematic of a fourth embodiment of an ophthalmic device with a laser apparatus according to the invention in an arrangement below a surgical microscope with a fixation light;

FIG. 5A shows a laser apparatus in a first variant with a ring projection system in a first configuration;

FIG. 5B shows the laser apparatus from FIG. 5A in a second configuration;

FIG. 6A shows a laser apparatus in a second variant with a ring projection system in a first configuration;

FIG. 6B shows the laser apparatus from FIG. 6A in a second configuration;

FIG. 7 is a schematic of a fifth embodiment of an ophthalmic device with a laser apparatus in accordance with FIGS. 6A and 6B in an arrangement below a surgical microscope; and,

FIG. 8 is a schematic illustration of a sixth embodiment of an ophthalmic device with a laser apparatus in accordance with FIGS. 6A and 6B in an arrangement in a surgical microscope.

### DESCRIPTION OF THE PREFERRED EMBODIMENTS OF THE INVENTION

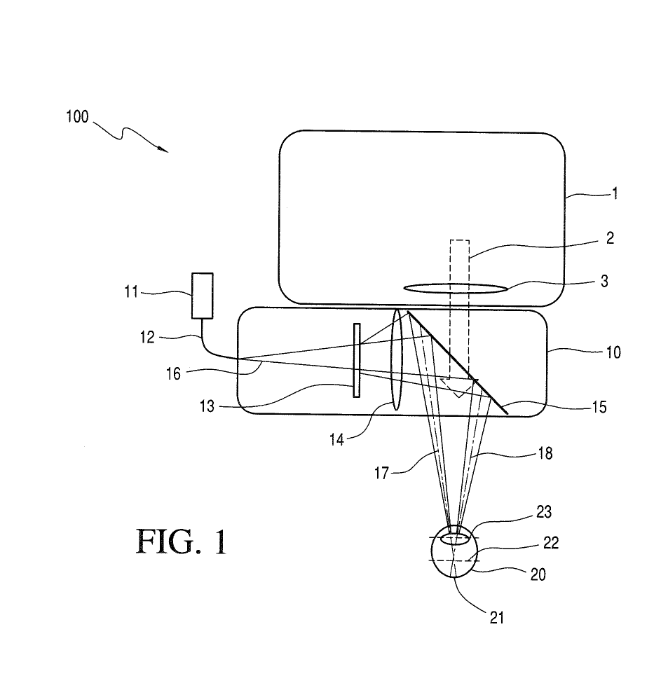

FIG. 1 is a schematic of a first embodiment of an ophthalmic device 100 with a laser apparatus 10 according to the invention in an arrangement below a surgical microscope 1.

The surgical microscope 1 has a viewing beam path 2, which is guided through a main objective 3.

The laser apparatus 10 is arranged below the surgical microscope 1. The laser apparatus 10 includes a laser 11, an optical waveguide 12, an optical beam deflection device 13, imaging optics 14 and an in-coupling element 15.

The light emerging from the laser 11 is coupled into the optical waveguide 12. The light 16 emerging from the optical waveguide 12 is incident on the optical beam deflection device 13, which generates a variable line contour, for example, a ring-shaped or oval line contour, from the punctiform laser light.

The laser light in the form of a line contour is guided through imaging optics 14 and coupled into the viewing beam path 2 via the in-coupling element 15. The two beam paths of the line contour reflected at the in-coupling element 15 are schematically depicted by the beam paths (17, 18). The laser light in the form of a line contour is focused by the imaging optics in a focal plane 23. The focal plane 23 lies in the region of the anterior capsular bag membrane of an eye 20.

The optical beam deflection device 13 and the imaging optics 14 shape the light of the laser light beam in such a way that the line focus is formed in a focal plane 23. The cutting process in the tissue region of the eye 20 is performed in the region of the focal plane 23.

The beam paths (17, 18) intersect in a pupil plane 22 within the vitreous humor of the eye 20. As a result, no laser light is incident on a macula 21.

The surgical microscope 1 can have a monoscopic or stereoscopic embodiment and it has further optical elements. The surgical microscope can have a zoom system, eyepieces, data feed-in and/or a camera system. The surgical microscope can be configured for a single observer or for two or more observers.

The light source 11 is a laser light source, which emits yellow light in the wavelength range between 525 nm and 675 nm, for example in the wavelength range between 590 and 610 nm.

The optical waveguide 12 can include an optical fiber or a plastic optical waveguide. It is conceivable to embody the optical waveguide 12 as a monomode fiber or multimode fiber, wherein the optical waveguide 12 is suitable for transmitting the laser-light power for the purposes of treating the eye tissue.

The beam deflection device 13 can be any optical element which has a contour-shaping optical property. By way of example, it can be embodied as a convex or concave axicon lens element, as a lens system or as a micro-mirror array. Any line contour can be generated by the beam deflection device 13. The contour can have a line-shaped, straight, curved, closed or open configuration. The line contour can have continuous or interrupted structures. A ring contour, an oval contour, a free-form contour or a cross-shaped contour is conceivable. The contour shaping element can also include a plurality of optical elements.

If the contour-shaping element of the beam deflection device 13 is an axicon lens element, the size of the line contour can be varied by virtue of the axicon lens element being displaced along the optical axis thereof. If the beam deflection device 13 is embodied as a micro-mirror array, the shape and size of the line contour can be varied by selective switching of individual micro-mirrors.

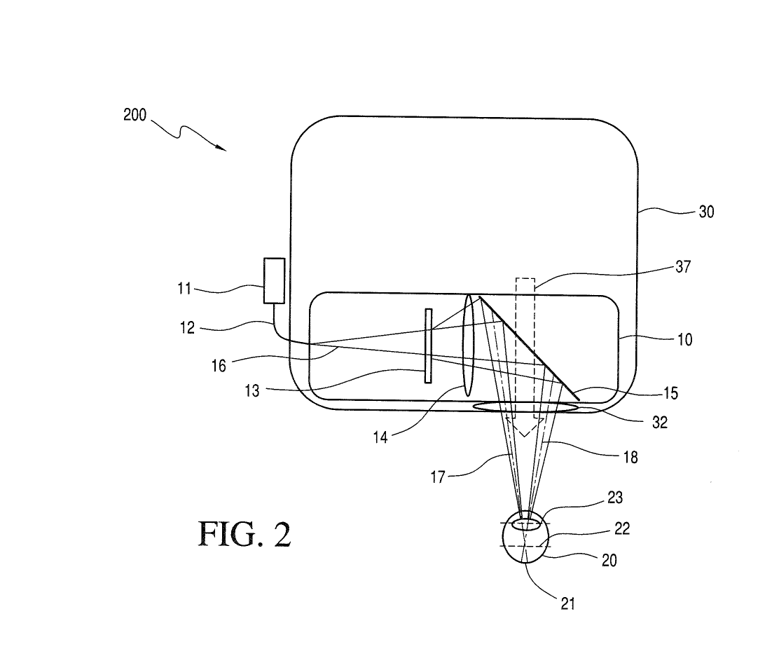

FIG. 2 is a schematic illustration of a second embodiment of an ophthalmic device 200 with the laser apparatus 10 according to the invention in an arrangement in a surgical microscope 30.

The laser apparatus 10 has the same components that were already described in FIG. 1. However, this embodiment distinguishes itself by virtue of the laser apparatus 10 with the in-coupling element 15 being arranged above a main objective 32. The laser line contour, schematically depicted by the two beam paths (17, 18), coupled into an observation beam path 31 is therefore guided to the eye 20 through the main objective 32.

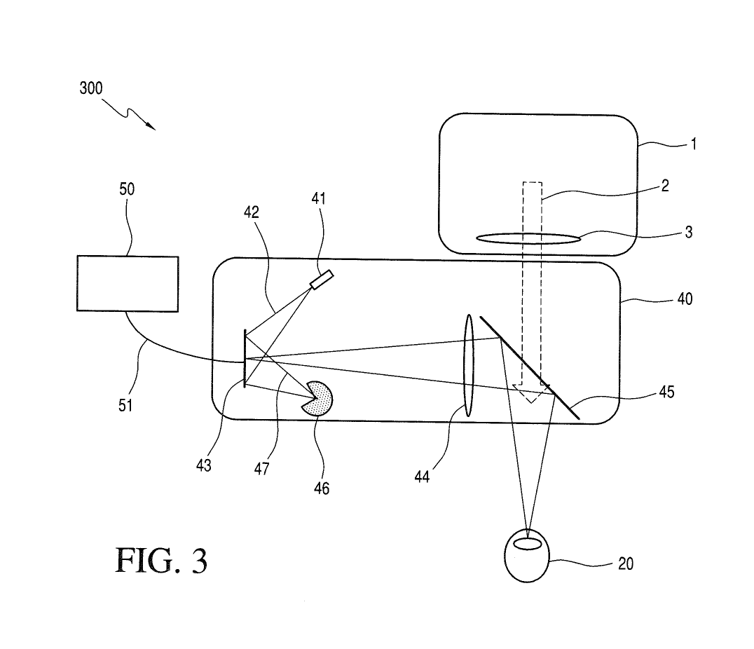

FIG. 3 shows a schematic illustration of a third embodiment of an ophthalmic device 300 with a laser apparatus 40 according to the invention in an arrangement below a surgical microscope 1 with a micro-mirror array 43.

The light 42 emerging from a laser 41 is incident on the micro-mirror array 43 as a contour-shaping beam deflection device. The micro-mirror array is connected to a control unit 50 via a line 51. The control unit 50 is configured to set the reflection direction of the individual micro-mirrors. Some of the micro-mirrors are set in such a way that the laser light, for example in the form of a ring-shaped or oval line contour, is guided to the eye 20 via imaging optics 44 and an in-coupling element 45. Laser light 47 that is not guided to the eye 20 is deflected to a light trap 46 by the micro-mirror array.

The micro-mirror array 43 can have a matrix of 1000×1000 micro-mirrors. Advantageously, any desired free-form contour can be projected as a line focus with high resolution onto the eye 20 by way of a micro-mirror array. Closed and interrupted contours are possible. It is also possible to project a plurality of contours onto the eye 20 simultaneously, for example circles and lines together.

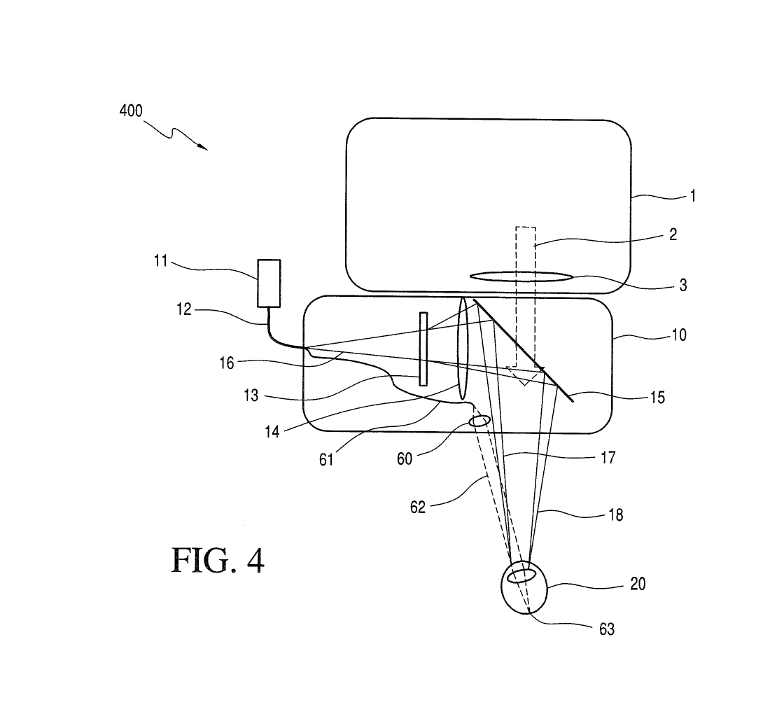

FIG. 4 shows a schematic illustration of a fourth embodiment of an ophthalmic device 400 with a laser apparatus 10 according to the invention in an arrangement below a surgical microscope 1 with a fixation light.

The laser apparatus 10 has the same components that are described in FIG. 1. However, this embodiment distinguishes itself by virtue of a small portion of the laser light emanating from the optical waveguide 12 being guided to a lens element 60 by way of an optical waveguide 61. The lens element 60 is embodied as a converging lens element and images the light beam emerging from the optical waveguide 61 at infinity, depicted by the beam path 62. This light forms a fixation light for the eye 20. The fixation light and the line focus can be switched on and off independently of one another via shutter elements or an appropriately actuated micro-mirror array. The eye 20 is directed to the fixation light and it is therefore situated in an inclined position in relation to the optical axis of the viewing beam path 2. The fixation light, which has a non-hazardous light power for the eye, is imaged on the macula 63.

As a result, the location of the patient eye is in a defined and stable position during the tissue treatment. The laser contour to be projected can easily be positioned on the eye. Then, no laser light from the line focus can be incident on the macula 63.

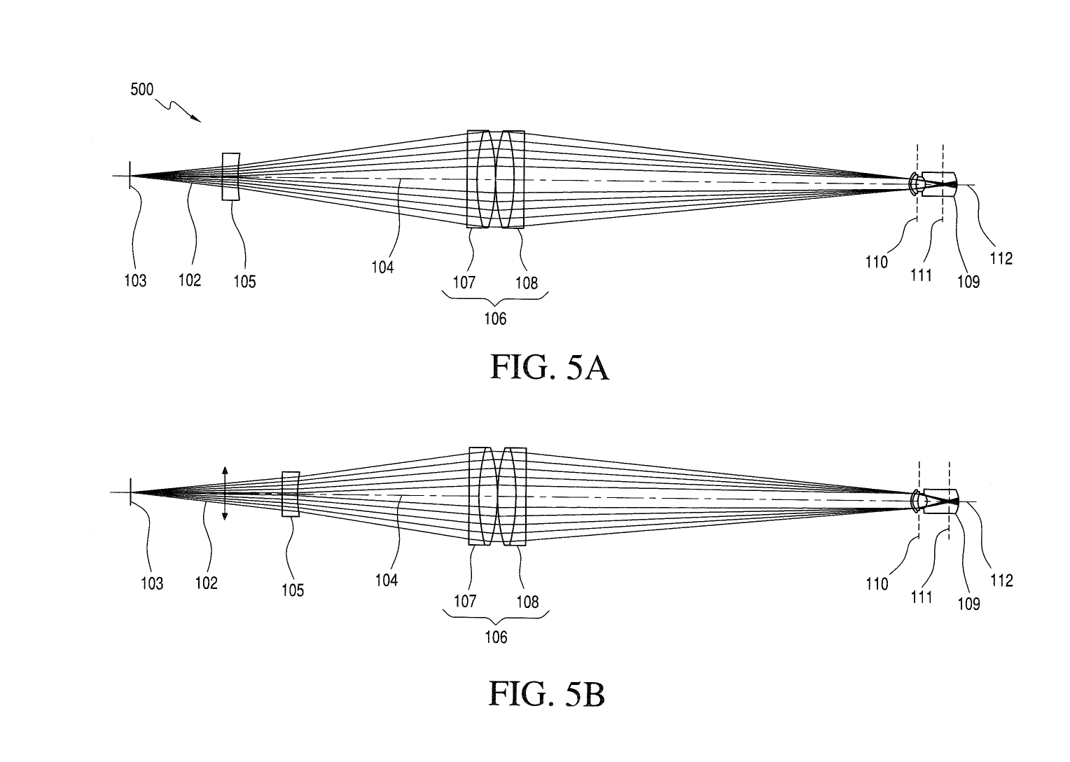

FIG. 5A shows a laser apparatus 500 in a first variant with a ring projection system in a first configuration.

The laser apparatus 500 is depicted without deflection by an in-coupling element. The laser apparatus 500 includes a laser with an outlet surface 103, at which a narrowband laser beam 102 with a compact cross section is emitted. The cross section of the laser beam 102 after the emergence from the laser can have an, for example, approximately round, rectangular or oval configuration.

The laser apparatus 500 furthermore includes a beam deflection device in the form of an axicon 105 with a concave or planoconcave configuration, the optical axis 104 of which is disposed in the laser beam 102. The concave axicon 105 effects a deflection of the laser beam away from the optical axis 104 and a reshaping of the cross section of the laser beam 102. In the present embodiment, a round cross section of the laser beam 102 on entry into the axicon 105 is assumed below, which cross section is reshaped into a ring-shaped cross section by the axicon.

In the further course, the laser beam with a ring-shaped cross section passes through imaging optics 106 which, in this embodiment, are formed by two cemented elements (107, 108) with positive refractive power. The imaging optics 106 can also include a differently embodied lens system with positive refractive power. The imaging optics 106 can also have a single converging lens element.

The main objective can form part of the imaging optics 106 in an embodiment in which the laser apparatus is arranged in a surgical microscope and the laser beam with a ring-shaped cross section is guided through the main objective of the surgical microscope.

The laser beam with a ring-shaped cross section is imaged in a focal plane 110 such that a ring-shaped line focus is formed in the focal plane 110. By way of example, the ring diameter of the line focus is configured to be between 3 mm and 5 mm.

A dye with a light-absorbing effect for the wavelength range of the laser is introduced into the tissue in the tissue region of an eye 109 which lies in the region of the focal plane 110. The dye can be trypan blue, which has a maximum light-absorbing effect in the tissue region at a wavelength of 599 nm.

The dye trypan blue as a base has a maximum light-absorbing effect at 591 nm. However, if the dye is introduced into the tissue region of the eye, the maximum light-absorbing effect in the tissue region shifts to a wavelength of 599 nm.

In the example, the laser is embodied to emit a narrowband light beam within a wavelength range of 590 nm to 610 nm and it has an emission power maximum at 599 nm.

The laser light is directed onto the focal plane 110 as a ring-shaped line focus within a very short period of time, for example, 200 ms or 500 ms, and thus brings about a circular cut in the tissue, for example, in the capsular bag. Thus, the laser apparatus enables the treatment of tissue in the anterior of an eye in the region of the focal plane 110.

By coloring the tissue region with the trypan blue dye and by matching the laser light to the wavelength range of the dye in the tissue, it is possible to carry out the cut with a relatively low laser power per unit area, for example with a laser power per unit area of 2×10⁴ W/cm².

As a result of the special shaping of the laser beam, it is possible to carry out the tissue treatment in a single work step and not in a sequence, as is the case in a scanning method.

The light beams of the laser cross in a pupil plane 111 in the vitreous humor of the eye 109. The pupil plane 111 lies in front of the region of the macula 112, and so the macula 112 is not illuminated by laser light.

FIG. 5B shows the laser apparatus from FIG. 5A in a second configuration.

In the second configuration, the concave axicon 105 is displaced along the optical axis 104 in the direction toward the imaging optics 106. This brings about a widening of the ring diameter in the focal plane 110. By way of example, the ring diameter of the line focus in the focal plane 110 is configured to be between 5 mm and 8 mm.

The light beams of the laser cross in a pupil plane 113 in the vitreous humor of the eye 109. The pupil plane 113 lies in front of the region of the macula 112, and so the macula 112 is not illuminated by laser light.

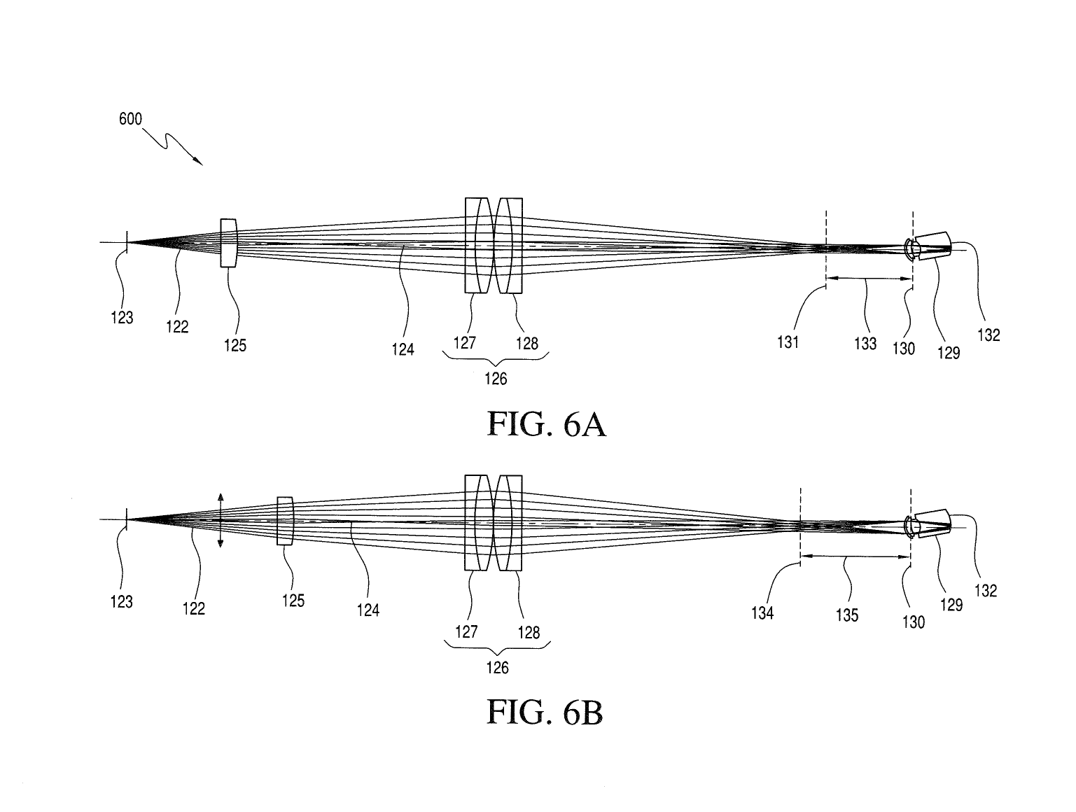

FIG. 6A shows a laser apparatus 600 in a second variant with a ring projection system in a first configuration.

The laser apparatus 600 is depicted without deflection by a coupling element. The laser apparatus 600 includes a laser with an emergence surface 123. The laser apparatus 500 furthermore includes a beam deflection device in the form of an axicon 125 with a convex or planoconvex embodiment, the optical axis 124 of which is arranged in the laser beam 122. The axicon 125 brings about a reshaping of the laser beam 122 into a ring-shaped cross section. In the further course, the laser beam with a ring-shaped cross section passes through imaging optics 126 which, in this embodiment, are formed by two cemented elements (127, 128) with positive refractive power.

The laser beam with a ring-shaped cross section is imaged in a focal plane 130 such that a ring-shaped line focus is formed in a focal plane 130. In this example, the ring diameter of the line focus is 4.6 mm. A dye has been introduced into the tissue in the tissue region of an eye 129 lying in the region of the focal plane 130, the dye having a light-absorbing effect for the wavelength range of the laser. Thus, the laser light brings about a circular cut in the tissue of the eye 129 in the region of the focal plane 130.

In this second variant, the light beams of the laser are guided closer to the optical axis 124 when passing through the imaging optics 126 than in the first variant in accordance with FIGS. 5A and 5B. This is advantageous in that smaller optical elements can be used in the imaging optics 126. In order nevertheless to achieve a suitable angle of incidence of the light beams in the focal plane 130 of the eye, the light beams of the laser are guided in such a way that they cross in a pupil plane 131 upstream of the eye 129. A distance 133 between the pupil plane 131 and the focal plane 130 typically lies in a region of between 10 mm and 50 mm.

In this variant, the eye 129 is arranged in a rotated manner in relation to the optical axis 124 such that the macula 132 is not illuminated by laser light.

FIG. 6B shows the laser apparatus from FIG. 6A in a second configuration.

In the second configuration, the convex axicon 125 is displaced along the optical axis 124 in the direction toward the imaging optics 126. This brings about a widening of the ring diameter in the focal plane 130. By way of example, the ring diameter of the line focus is configured to be between 5 mm and 8 mm. The position of the pupil plane 134 is displaced in the direction toward the imaging optics 126. This results in a greater distance 135 between the focal plane 130 and the pupil plane 134.

The eye 129 is arranged in a rotated manner in relation to the optical axis 124 such that the macula 132 is not illuminated by laser light.

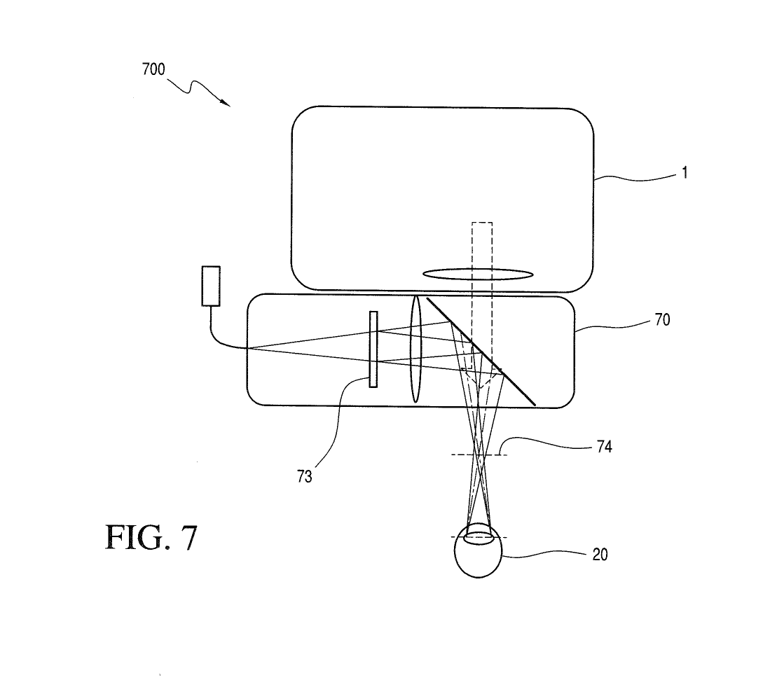

FIG. 7 is a schematic illustration of a fifth embodiment of an ophthalmic device 700 with a laser apparatus 70 in accordance with FIGS. 6A and 6B in an arrangement below a surgical microscope 1.

The ophthalmic device 700 is embodied like the ophthalmic device 100 in accordance with FIG. 1, with the difference being that a beam deflection device 73 is embodied in such a way that the light beams of the laser cross in a pupil plane 74 upstream of the eye 20.

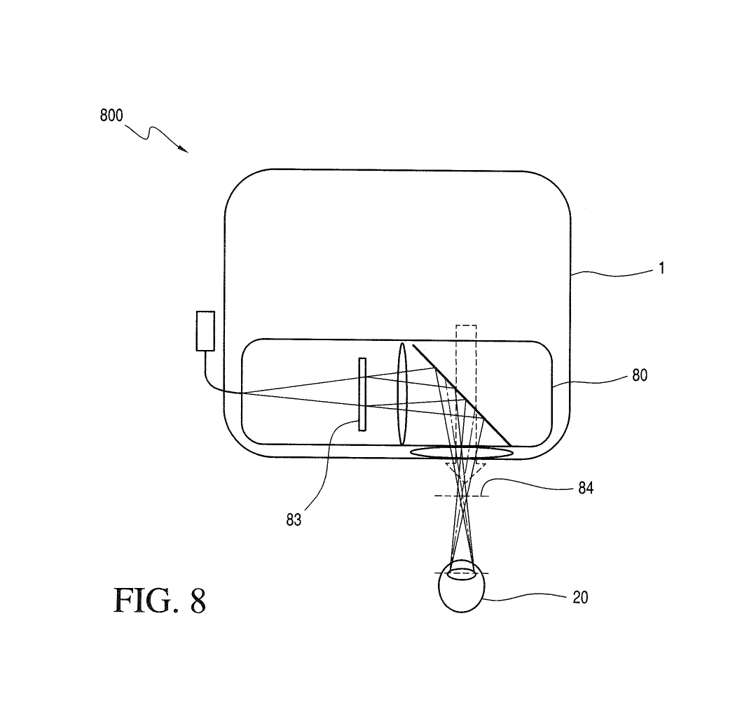

FIG. 8 is a schematic illustration of a sixth embodiment of an ophthalmic device 800 with a laser apparatus 80 in accordance with FIGS. 6A and 6B in an arrangement in a surgical microscope 1.

The ophthalmic device 800 is embodied like the ophthalmic device 200 in accordance with FIG. 2, with the difference being that a beam deflection device 83 is embodied in such a way that the light beams of the laser cross in a pupil plane 84 upstream of the eye 20.

It is understood that the foregoing description is that of the preferred embodiments of the invention and that various changes and modifications may be made thereto without departing from the spirit and scope of the invention as defined in the appended claims.

What is claimed is:

1. An ophthalmic apparatus for treating tissue in the anterior of an eye, the ophthalmic apparatus comprising:
a laser configured to generate a light beam;
an optical arrangement configured to reshape said light beam into a line focus having a length to width ratio of at least 10;
an optical system configured to guide said light beam to an object plane configured to have the tissue to be treated arranged therein; and,
said laser being configured to emit yellow light in a first wavelength range of 525 nm to 675 nm.

2. The ophthalmic apparatus of claim 1, wherein:
said line focus has an image in said object plane; and,
a numerical aperture and said image in said object plane are matched to each other in such a manner so as to cause an energy density in said object plane having a magnitude of less than 10⁶ W/cm².

3. The ophthalmic apparatus of claim 1, wherein said optical arrangement includes one of the following: a concave axicon, a convex axicon, a diffractive optical element, a reflection echelon grating and a micro-mirror array.

4. The ophthalmic apparatus of claim 1, wherein said laser has a power maximum at a wavelength of 599 nm.

5. The ophthalmic apparatus of claim 1 further comprising an observation unit.

6. The ophthalmic apparatus of claim 5, wherein:
said observation unit defines a viewing beam path; and,
said observation unit includes a main objective and an in-coupling element configured to couple said light beam into said viewing beam path.

7. The ophthalmic apparatus of claim 6, wherein:
said in-coupling element is configured as a reflection-bandpass-filter having a bandpass transmission value T>95% for a wavelength lying in a range given by 599 nm±10 nm.

8. The ophthalmic apparatus of claim 5, wherein said in-coupling element is arranged above said main objective.

9. The ophthalmic apparatus of claim 5, wherein said observation unit has one of an ambient illumination and a coaxial illumination from which a fixation light is decoupled.

10. The ophthalmic apparatus of claim 1, wherein a fixation light is decoupled from said light beam of said laser.

11. The ophthalmic apparatus of claim 1, wherein said length to width ratio is at least 100.

12. The ophthalmic apparatus of claim 1, wherein said length to width ratio is at least 1000.

13. The ophthalmic apparatus of claim 1, wherein said first wavelength range is 550 nm to 610 nm.

14. The ophthalmic apparatus of claim 1, wherein said first wavelength range is 580 nm to 610 nm.

15. The ophthalmic apparatus of claim 2, wherein said energy density in said object plane has a magnitude of less than 10⁵ W/cm².

16. The ophthalmic apparatus of claim 2, wherein said energy density in said object plane has a magnitude of less than 10⁴ W/cm².

17. A method for thermal treatment of a tissue in the anterior of an eye using an ophthalmic apparatus for treating tissue in the anterior of an eye, the ophthalmic device including a laser configured to generate a light beam; an optical arrangement configured to reshape said light beam into a line focus having a length to width ratio of at least 10; an optical system configured to guide said light beam to an object plane configured to have the tissue to be treated arranged therein; and, said laser being configured to emit yellow light in a wavelength range of 525 nm to 675 nm, the method comprising the step of:
introducing a dye into the object plane of the tissue region, the dye having a light absorbing effect for said wavelength range of said laser.

18. The method of claim 17, wherein the dye is trypan blue dye having a maximum light absorbing effect in the tissue at a wavelength of 599 nm.
